(19) United States
(12) Patent Application Publication
(10) Pub. No.: US 2011/0066134 A1
(43) Pub. Date: Mar. 17, 2011

(54) Controlling Drug Transport and Current in Iontophoretic Onychomycosis Treatment

(75) Inventors: Michael Barsness, Oxford, MA (US); Shawn Davis, Boston, MA (US); Robert Etheredge, Natick, MA (US); Kuowei Chang, Lexington, MA (US); Hyun Kim, Weston, MA (US)

(73) Assignee: Nitric Biotherapeutics, Inc.

(21) Appl. No.: 12/755,989

(22) Filed: Apr. 7, 2010

Related U.S. Application Data
(60) Provisional application No. 61/167,261, filed on Apr. 7, 2009.

(51) Int. Cl. A61N 1/30

(52) U.S. Cl. 604/501

(57) ABSTRACT

Pharmaceutical drugs and formulations suitable for iontophoresis thereof that provide enhanced iontophoretic delivery of an anti-fungal drug to at least one body surface, such as a nail, are described. Also described are pharmaceutical formulations suitable for iontophoresis comprising terbinafine and methods for administering terbinafine to a body surface via iontophoresis. When the drug source is introduced into the nail using a mask, terbinafine concentrations in peripheral areas of nail following iontophoretic treatment are less than drug concentrations directly under the mask. Further, differences in delivered drug concentrations is effectuated by creating drug delivery gradients. Drug can be delivered to dissimilar tissues based on different transfer efficiencies exhibited by the dissimilar tissues, resulting in higher percentage delivery in one tissue vs. another tissue.

### CROSS-REFERENCE TO RELATED APPLICATIONS

This application claims the benefit of priority to U.S. Provisional Patent Application No. 61/167,261 filed Apr. 7, 2009, the entire disclosure of which is incorporated by reference herein as if set forth herein in its entirety.

### BACKGROUND OF THE INVENTION

Onychomycosis is a disease of the nail caused by yeast, dermatophytes, or other molds, effecting both fingernails and toenails and characterized by discoloration, onycholysis, and accumulation of subungual debris and nail plate dystrophy. The disease adversely affects the quality of life of its victims, with subject complaints ranging from unsightly nails and discomfort with footwear, to more serious complications including secondary bacterial infections.

Because nail fungal infections reside in an area difficult to access by conventional topical treatment, anti-fungal drugs cannot readily penetrate the nail plate to reach the infection sites under the nail. Therefore, onychomycosis has traditionally been treated by oral administration of anti-fungal drugs. However, this leads to the potential for side effects of such drugs, in particular those side effects caused by more potent anti-fungal drugs. Alternatively, onychomycosis has been treated by removal of the nail before treating with a topically active anti-fungal agent, but this also creates an equally undesirable effect.

Iontophoresis has been known for many years, as a means to deliver drugs and cosmetic active agents into the skin for therapeutic purposes. An iontophoretic delivery system is, for example, a drug delivery system that releases drug at a controlled rate to the target tissue upon application. The advantages of systems wherein drug is delivered locally via iontophoresis are the ease of use, relatively safe administration, the ability to finely modulate the dose by changing the time of application and/or the current level and the ability to interrupt administration by simply stopping the current and/or peeling off or removing it from the skin or other body surface whenever an overdosing is suspected. In recent years iontophoretic delivery of drugs has attracted wide attention as a better way of administering drugs for local as well as systemic effects. The design of iontophoretic delivery systems can usually be such that the side effects generally seen with the systemic administration of conventional dosage forms are minimized.

Iontophoresis involves the application of an electromotive force to drive or repel ions through the dermal layers into a target tissue. Particularly suitable target tissues include those adjacent to the delivery site for localized treatment. Uncharged molecules can also be delivered using iontophoresis via a process called electroosmosis.

Regardless of the charge of the medicament to be administered, an iontophoretic delivery device employs two electrodes (an anode and a cathode) in conjunction with the patient's skin to form a closed circuit between one of the electrodes (referred to herein alternatively as a “working” or “application” or “applicator” electrode) which is positioned at the site of drug delivery and a passive or “grounding” electrode affixed to a second site on the skin to enhance the rate of penetration of the medicament into the skin adjacent to the applicator electrode. U.S. Pat. No. 6,477,410 issued to Henley et al. describes the use of iontophoresis for drug delivery in the treatment of a variety of diseases, such as onychomycosis.

Traditionally, iontophoretic drug delivery has been applied to a single, living tissue type, e.g. stratum corneum and dermis. Iontophoretic treatment of onychomycosis employs this technology to deliver drug actives into saline soaked nail, nail bed and other soft tissues. These tissue types are significantly different. Models reported in the literature have predominantly focused on single tissue type delivery. Those models take into account the complex electro-chemical and physiologic factors that influence drug flow in that “relatively” homogenous environment. It is perhaps not unexpected that drug delivery into soft tissue alone at one efficiency level might not be the same as drug delivery into nail & nail bed.

Therefore, there is a long felt need in the art for an iontophoretic treatment system for onychomycosis, using drug applicators targeting either toe nail only or nail and surrounding tissue, where the amount of drug delivered is regulated based on drug delivery gradients and/or calculated transfer efficiencies of the targeted tissue.

### SUMMARY OF THE INVENTION

The present invention relates to a method for treating a fungal infection of the nail of a patient suffering therefrom, comprising iontophoretically administering terbinafine to at least a portion of the infected nail, wherein the amount of terbinafine delivered is based upon a drug delivery gradient across the infected nail.

In one embodiment, the iontophoretic administration comprises a mask for limiting the nail surface to which terbinafine is iontophoretically administered. In another embodiment, the drug delivery gradient is calculated prior to administration of terbinafine. In another embodiment, the amount of current used to iontophoretically administer terbinafine is based on the calculated drug delivery gradient. In another embodiment, the length of time and frequency of iontophoretic administration is based on the amount of current used. In another embodiment, the terbinafine is a component of a formulation comprising terbinafine hydrochloride. In another embodiment, the formulation further comprises one or more solvents or co-solvents and a permeation enhancer. In another embodiment, the terbinafine hydrochloride is present in an amount from about 1 to about 10% (w/w). In another embodiment, the one or more solvent or co-solvents are each present in an amount from about 10 to about 50% (w/w). In another embodiment, the formulation further comprises water, an alcohol and glycerin.

The present invention also relates to a method for treating a fungal infection of the nail of a patient suffering therefrom, comprising iontophoretically administering terbinafine to at least a portion of the nail and at least a portion of tissue adjacent the nail, wherein the amount of terbinafine delivered to the nail and adjacent nail tissue is based upon different transfer efficiencies of the nail and adjacent tissue, such that the nail receives a higher percentage delivery of terbinafine than the adjacent tissue.

In one embodiment, the different transfer efficiencies of the nail and adjacent tissue is calculated prior to administration of terbinafine. In another embodiment, the amount of current used to iontophoretically administer terbinafine is based on the calculated transfer efficiencies of the nail and adjacent tissue. In another embodiment, the length of time and frequency of iontophoretic administration is based on the amount of current used. In another embodiment, the terbinafine is a component of a formulation comprising terbinafine hydrochloride. In another embodiment, the formulation further comprises one or more solvents or co-solvents and a permeation enhancer. In another embodiment, the terbinafine hydrochloride is present in an amount from about 1 to about 10% (w/w). In another embodiment, the one or more solvent or co-solvents are each present in an amount from about 10 to about 50% (w/w). In another embodiment, the formulation further comprises water, an alcohol and glycerin. In another embodiment, the ratio of the different transfer efficiencies in the nail and adjacent nail tissue are equal to or greater than about 2 to 1.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 depicts a current test fixture for measuring current distributions. The device is designed to support cadaver nail over agarose (skin analogue) and measure current distribution in two dimensions during iontophoresis using masked drape and full drape applicator designs. The device measures and records current flow in real time using a 45 node sensor with 3 mm spacing and associated data acquisition system. The overall fixture covers approximately a 12-20 mm diameter circle.

FIG. 2, comprising FIGS. 2A and 2B, depicts an iontophoretic power source and an applicator, respectively. The power source of FIG. 2A may be used in the treatment of onychomycosis. The applicator as depicted in FIG. 2B is a full drape embodiment. A masked drape embodiment would include the mask between the applicator and the nail.

FIG. 3, comprising FIGS. 3A and 3B, demonstrates assay results from cadaver nails following iontophoresis using a masked drape applicator. FIG. 3A depicts drug delivery, while FIG. 3B depicts current measurements.

FIG. 4 depicts a current distribution parallel resistance model for masked drape applications.

FIG. 5 depicts a graph of drug assay vs. resistance model for masked drape applications.

FIG. 6 depicts current measurements in a full drape application.

FIG. 7, comprising FIGS. 7A and 7B, depicts clinical voltages for masked and full drape applications (FIG. 7A), and average nail and skin “resistance” over time (FIG. 7B).

FIG. 8 is a graph depicting terbinafine levels in skin vs. nail and nail bed of cadaver toes following iontophoresis at about 0.5 mA for 20 min.

FIG. 9 is a graph depicting aggregate transport number for masked and full drape applications.

FIG. 10 is a graph depicting the projected delivery of terbinafine in skin and nail.

### DETAILED DESCRIPTION

The present invention provides an iontophoretic treatment system for onychomycosis, using drug applicators targeting either nail only or nail and surrounding tissue. The present invention is based on the discovery that terbinafine concentrations in peripheral areas of nail following iontophoretic treatment, where the drug source is introduced into the nail using a mask, were shown to be less than drug concentrations directly under the mask due to current and drug delivery gradients. Terbinafine concentrations resulting from simultaneous iontophoretic delivery to dissimilar tissues were also found to exhibit different transfer efficiencies, resulting in higher percentage delivery in nail than soft tissue. Therefore, the present invention includes an iontophoretic treatment system and method for onychomycosis, using drug applicators targeting either nail only or nail and surrounding tissue, where the amount of drug delivered is regulated based on calculated drug delivery gradients and/or transfer efficiencies of the targeted tissue.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein, an “antifungal drug” is a drug that directly or indirectly inhibits or reduces fungal growth, contamination or infection, or a symptom or condition associated with fungal growth, contamination or infection, or that decreases or reduces recurrence of or susceptibility to fungal growth, contamination or infection by a fungus, regardless of the mode of action.

As used herein, a “nail” includes reference to the whole nail or any portion of the nail, including the nail plate, the nail bed, the cuticle, the nail folds, the lunula, the matrix, and the hyponychium.

As used herein, a “permeation enhancer” or “penetration enhancer” is a material which achieves permeation enhancement or an increase in the permeability of the skin and/or nail to a pharmacologically active agent.

As used herein, the term “pharmaceutically acceptable carrier or excipient” means any non-toxic diluent or other formulation auxiliary that is suitable for use in iontophoresis.

As used herein, a “therapeutically effective amount” is an amount of drug, such as an anti-fungal drug, that is sufficient to prevent development of or alleviate to some extent one or more of a patient's symptoms of the disease being treated.

### DESCRIPTION

The present invention provides an iontophoretic treatment system for onychomycosis, using drug applicators targeting either nail only or nail and surrounding tissue. The present invention is based on the discovery that terbinafine concentrations in peripheral areas of nail following iontophoretic treatment, where the drug source is introduced into the nail using a mask, were shown to be less than drug concentrations directly under the mask due to current and drug delivery gradients. Terbinafine concentrations resulting from simultaneous iontophoretic delivery to dissimilar tissues were also found to exhibit different transfer efficiencies, resulting in higher percentage delivery in nail than soft tissue. Notably, for terbinafine into nail and nail bed vs. surrounding soft tissue, transfer efficiencies were found to be more than about 2 to 1 in cadaver toes in favor of nail per mA-min (p=0.009).

As contemplated and demonstrated herein, clinical data illustrate that levels of drug delivery differ unexpectedly from relative dosing levels to multiple tissue types, using current monitoring and analysis techniques, coupled with assays of drug delivery into excised nail and cadaver toe. The results provided herein indicate significant correlation with piecewise linear models of current flow and extracted drug in the nail-only application. Thus, for nail and surrounding tissue applications, drug load per unit dose (mA-min) is more efficient into nail than into surrounding tissue (2.38:1 ug/mA-min nail vs. surrounding tissue, n=6, p=0.009).

The present invention includes iontophoretic systems and methods useful for topically treating onychomycosis, a disease, or fungal infection, of the nail plate on the hands or feet. As used herein, a “nail” includes reference to the whole nail or any portion of the nail, including the nail plate, the nail bed, the cuticle, the nail folds, the lunula, the matrix, and the hyponychium. The present invention also includes a device that delivers a therapeutic agent to and through the nail plate and to the nail bed. In one embodiment, the compound is an anti-fungal agent, such as terbinafine. In certain embodiments, the anti-fungal agent is delivered to the nail only or the nail and surrounding tissue. In an exemplary embodiment of the present invention, an iontophoretic device is used to facilitate iontophoretic delivery of an anti-fungal agent into and through the nail plate.

### Applicator Designs

As contemplated herein, the present invention includes a drug applicator that can be used with a mask (or masked drape) to limit contact and delivery to a portion of the nail and the nail bed beneath, or an applicator that is used without a mask (or full drape) to deliver drug to the nail, nail bed and surrounding soft tissue.

The applicator may include a flexible, wearable patch that can conform to a nail surface or a portion of the nail surface. The applicator may be sized so as to be applied to the nail of one digit, or it may be sized so as to be applied to a plurality of digits, such as by wrapping the device around two digits that are next to one another. The applicator can be fabricated from thin and flexible materials, which enable at least those surfaces that contact a patients nail or skin to conform to the contours of the patient when the applicator is applied thereon. The applicator may be provided with an adhesive that allows it to adhere to the nail surface and/or surrounding tissue of the nail. Additional examples of iontophoretic delivery devices useful with the compositions and methods described herein include, but are not limited to, handheld devices and devices which comprise a separate compartment as a power supply. Exemplary devices include, but are not limited to, those described in U.S. Pat. Nos. 6,148,231, 6,385,487, 6,477,410, 6,553,253, and U.S. Patent Publication Numbers 2008/0319371, 2004/0111051, 2003/0199808, 2004/0039328, 2002/0161324, and U.S. Application Ser. No. 60/743,528, all incorporated herein by reference herein in their entirety.

An example of an applicator which can be used with terbinafine or a terbinafine formulation comprises an active electrode adhered to an open cell polymer foam or hydrogel. Medicament layers may form part of the applicator or may alternatively remain a separate component from the applicator, such that the applicator may be applied adjacent to, on top of or underneath the medicament layer. Another applicator which has been developed for use with a device for iontophoretic delivery of an agent to a treatment site comprises an applicator head having opposite faces and including an active electrode and a porous pad (such as a woven or non-woven polymer, for example, a polypropylene pad); a margin of the applicator head about the active electrode having a plurality of spaced projections there along; the porous pad and the applicator head being ultrasonically welded to one another about the margin of the head with the electrode underlying the porous pad; and a medicament or a medicament and an electrically conductive carrier therefor carried by the porous pad in electrical contact with the electrode. In one embodiment, the device includes carbon electrodes, silver-silver chloride electrodes or silver coated carbon electrodes. In a further embodiment, the applicator is a nail-only applicator wherein drug is delivered only to the nail. In another embodiment, the applicator is a nail and skin applicator wherein drug is delivered into the nail and into the surrounding skin. Of course, such applicators are preferably suitable for use in a masked or unmasked drape configuration.

### Anti-Fungals and Formulations

As contemplated herein, the system and method of the present invention may be used to deliver active agents, such as antifungal drugs, directly to and through the nail or the nail and surrounding tissues. Exemplary agents, drugs and formulations can be found in U.S. application Ser. No. 12/056, 802, the entire disclosure of which is incorporated by reference herein as if set forth in its entirety. Other exemplary agents can also be found in U.S. Pat. Nos. 6,148,231, 6,385,487, 6,477,410, 6,553,253, and U.S. Patent Publication Numbers 2008/0319371, 2004/0111051, 2003/0199808, 2004/0039328, 2002/0161324, and U.S. Application Ser. No. 60/743,528.

For example, classes of antifungal drugs suitable for use with the present invention include, but are not limited to, allylamines, azoles, pyrimidines, tetraenes, thiocarbamates, sulfonamides, glucan synthesis inhibitors. Allylamines include, for example, amorolfine, butenafine, naftifine and terbinafine. Azoles include, for example, ketoconazole, fluconazole, elubiol, econazole, econaxole, itraconazole, isoconazole, imidazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, flutrimazole. Polyenes include, for example, nystatin, pimaricin and amphotericin B. Pyrimidines include, for example, flucytosine. Tetraenes include, for example, natamycin. Thiocarbamates include, for example, tolnaftate. Sulfonamides include, for example, mafenidine. Other antifungal drugs include ciclopirox and ciclopirox olamine.

In one embodiment, the antifungal drug is an allylamine. In another embodiment, the antifungal drug is an azole. In another embodiment, the antifungal drug is selected from the group consisting of terbinafine, ketoconazole, econazole, ciclopirox, ciclopirox olamine, fluconazole, itraconazole and amorolfine. In yet another embodiment, the antifungal agent is selected from the group consisting of terbinafine, ciclopirox and ciclopirox olamine. In an additional embodiment, the antifungal agent is selected from the group consisting of terbinafine and ciclopirox olamine. In certain embodiments, the antifungal agent is terbinafine. In a further embodiment, the terbinafine is terbinafine hydrochloride. In additional embodiments, multiple drugs, or drug combinations, may be administered.

In one embodiment, the present invention is directed to a formulation suitable for iontophoresis comprising an aqueous composition of an antifungal drug, a pharmaceutically acceptable excipient or carrier and a non-ionic surfactant. In another embodiment, the invention is directed to a formulation comprising an aqueous composition of terbinafine, a pharmaceutically acceptable excipient or carrier and a non-ionic surfactant. In an additional embodiment, the formulation may contain counter ions that can make ion pairs with the ionized drug and improve delivery of the ionized drug. In another embodiment, the invention is directed to a formulation suitable for iontophoresis comprising an antifungal drug and one or more solvents and co-solvents. In yet another embodiment, the invention is directed to a formulation suitable for iontophoresis comprising an antifungal drug and a permeation enhancer. In a further embodiment, the invention is directed to a formulation suitable for iontophoresis comprising an antifungal drug, one or more solvents or co-solvents and a permeation enhancer. In an additional embodiment, the invention is directed to a formulation suitable for iontophoresis comprising an antifungal drug, one or more solvents or co-solvents, a permeation enhancer and a non-ionic surfactant.

In yet another embodiment, the formulation can comprise an anti-fungal drug and one or more chemical permeation or penetration enhancers. A “permeation enhancer” or “penetration enhancer” is a material which achieves permeation enhancement or an increase in the permeability of the skin and/or nail to a pharmacologically active agent. The terms “permeation enhancer” and “penetration enhancer” are used interchangeably herein. Examples of such permeation enhancers include, but are not limited to, N-acetylcysteine, urea, salicylic acid, linoleic acid, benzoic acid, oleic acid, cysteine hydrochloride, cyclodextrin, dimethyl sulfoxide, polyethylene glycol (PEG), polyvinylpyrolidone (PVP), dimyristoyl phosphatidylserine, saturated fatty acids (including, for example, stearic acid and palmitic acid) and combinations thereof. In one embodiment, the formulation comprises a permeation enhancer that is a keratolytic agent selected from the group consisting of benzoic acid, oleic acid, cysteine hydrochloride, N-acetylcysteine and urea. In an additional embodiment, the formulation comprises a permeation enhancer selected from the group consisting of polyethylene glycol and polyvinylpyrrolidone. In another embodiment, the formulation comprises a penetration enhancer selected from the group consisting of benzoic acid, polyethylene glycol, polyvinylpyrrolidone and combinations thereof. In certain embodiments, the permeation enhancer utilized is a permeation enhancer that enhances permeation into and through the nail to the nail bed or into the skin. The amount of permeation enhancer that is utilized is the amount that increases the permeation of terbinafine compared to permeation of terbinafine in the absence of the permeation enhancer. In one embodiment, the one or more permeation enhancers are each included in the composition in an amount from about 0.01 to about 50% (w/w).

In another embodiment, the formulation comprises a permeation enhancer selected from the group consisting of benzoic acid, oleic acid, salicylic acid, cysteine hydrochloride, N-acetylcysteine and urea wherein the permeation enhancer is included in an amount from about 0.05 to about 5.0% (w/w). In yet another embodiment, the formulation comprises a permeation enhancer selected from the group consisting of benzoic acid, oleic acid, salicylic acid, cysteine hydrochloride, N-acetylcysteine and urea wherein the permeation enhancer is included in an amount from about 0.05 to about 2.0% (w/w). In a further embodiment, the formulation comprises a permeation enhancer selected from the group consisting of benzoic acid, oleic acid, salicylic acid, cysteine hydrochloride, N-acetylcysteine and urea wherein the permeation enhancer is included in an amount from about 0.05 to about 1.0% (w/w). In yet another embodiment, the formulation comprises a permeation enhancer selected from the group consisting of benzoic acid, oleic acid, salicylic acid, cysteine hydrochloride, N-acetylcysteine and urea wherein the permeation enhancer is included in the composition in an amount from about 0.01 to about 5.0% and any and all whole or partial increments there between, relative to the amount of terbinafine in the composition.

In another embodiment, the formulation comprises polyethylene glycol. Polyethylene glycol may act as a permeation enhancer and/or a solvent or co-solvent as described below. As will be appreciated by one having skill in the art, polyethylene glycol of various molecular weights can be used in the inventive formulation, including, but not limited to polyethylene glycol (PEG) 200, PEG 400, PEG 600, PEG 1000 and PEG 3550. In another embodiment, the polyethylene glycol is PEG 400. In an additional embodiment, the formulation comprises polyethylene glycol wherein the polyethylene glycol is included in the formulation in an amount from about 10 to about 50% (w/w) and any and all whole or partial increments there between. In yet another embodiment, the formulation comprises polyethylene glycol wherein polyethylene glycol is included in the formulation in an amount from about 20 to about 50% (w/w) and any and all whole or partial increments there between. In a further embodiment, the formulation comprises polyethylene glycol and is included in the formulation from about 25 to about 45% (w/w) and any and all whole or partial increments there between. In an additional embodiment, the formulation comprises polyethylene glycol and polyethylene glycol is included in the formulation in amount of about 30% (w/w). In a further embodiment, the formulation comprises polyethylene glycol in an amount of about 40% (w/w).

In an additional embodiment, the formulation comprises polyvinylpyrrolidone (PVP). Polyvinylpyrrolidone may act as a permeation enhancer and/or a solvent or co-solvent as described below. As will be appreciated by one having ordinary skill in the art, polyvinylpyrrolidone of various molecular weights can be used in the inventive formulation. In one embodiment, the polyvinylpyrrolidone has a molecular weight from about 1000 to about 50,000 and any and all whole or partial increments there between. In another embodiment, the formulation comprises PVP and is included in the formulation in an amount from about 0.01 to about 10% (w/w) and any and all whole or partial increments there between. In yet another embodiment, the formulation comprises PVP and is included in the formulation in an amount from about 0.01 to about 5% (w/w) and any and all whole or partial increments there between. In a further embodiment, the formulation comprises PVP and is included in the formulation in an amount from about 1 to about 5% (w/w) and any and all whole or partial increments there between.

In another embodiment, the formulation comprises an antifungal drug and one or more solvents or co-solvents are selected from the group consisting of glycerin, an alcohol, glycol, a glycol monoether, a glycol diether, dimethylsulfoxide, caprolactam, dimethylisosorbide, isopropylidene glycerol, dimethylimidazolidinone, N-methyl-pyrrolidone-2, pyrrolidone-2, ethyl lactate, polyoxyethylenated C8-C10 glycerides, glyceryl laurate, dimethylacetamide, polyethylene glycol, polyvinylpyrrolidone and the like as well as combinations thereof. In yet another embodiment, the formulation comprises glycerin. In yet another embodiment, the formulation comprises an alcohol. Exemplary alcohols include, but are not limited to, ethanol, butanol, tert-butyl alcohol and benzyl alcohol. In one embodiment, the alcohol is ethanol. In a further embodiment, the formulation comprises glycerin and an alcohol. In an additional embodiment, the formulation comprises glycerin and ethanol.

The formulation can also contain a stabilizer such as an antioxidant or a chelating agent (including, for example, EDTA, disodium EDTA, butylated hydroxyl toluene, butylated hydroxy anisole, TPGS, sodium sulfites, ascorbic acid, vitamin E, etc.) and/or an alcohol (including, for example, ethyl alcohol). In another embodiment, the formulation may contain a preservative including, but not limited to, sodium benzoate, benzalkonium chloride, parabens (including methyl and propyl paraben), and the like. In a further embodiment, the formulation may contain an agent that affects protein binding including, but not limited to, linolenic acid, oleic acid, dimyristoyl phosphatidyl glycerol (DMPG), dimyristoyl phosphatidyl choline (DMPC) and isopropyl myristate.

As used herein, the term “pharmaceutically acceptable carrier or excipient” means any non-toxic diluent or other formulation auxiliary that is suitable for use in iontophoresis. Examples of pharmaceutically acceptable carriers or excipients include but are not limited to a solvent, co-solvents (including, for example, a solvent or co-solvent described above including, but not limited to, glycerin and/or an alcohol), a solubilizing agent (such as sorbitol or glycerin), a buffer, a pharmaceutically acceptable base, an alcohol such as benzyl alcohol and butanol and a viscosity modulating agent such as cellulose and its derivatives. The aqueous composition of terbinafine can comprise one or more solvents or co-solvents. In one embodiment, the solvent is water or a buffer. Buffers include, for example, phosphate buffer, citrate buffer, acetate buffer, piperazine-N,N′-bis(2-ethanesulfonic acid) (PIPES) buffer, dimethyl arsenate (Cacodylate) buffer and 2-(N-morpholino)ethanesulfonic acid (MES) buffer. In one embodiment, the buffer is a phosphate buffer. In another embodiment, the buffer is phosphate buffered saline.

In an additional embodiment, the antifungal agent is terbinafine and the pH of the formulation is less than about pH 7. In a further embodiment, the pH of the formulation is less than about 6. In an additional embodiment, the pH is less than about 5. In a further embodiment, the pH of the formulation is about 5. In another embodiment, the pH of the formulation is about 2.5 to about. 4.5 and any and all whole or partial increments there between. In a further embodiment, the pH of the formulation is about 3.

In another embodiment, the formulation is adsorbed into a foam material or used to swell a hydrogel. The foam material or hydrogel may be masked or unmasked, as discussed herein throughout.

In one embodiment, the viscosity of the formulation comprising terbinafine is similar to that of water. In another embodiment, the formulation is a liquid formulation.

Non-ionic surfactants can be included in the inventive formulation and include, for example, an ethoxylated polysorbate, polyethylene glycol, ethylene oxide/propylene oxide copolymer, and polyethoxylated castor oil. Ethoxylated polysorbates, include, for example, polysorbate-20 (Tween-20), polysorbate-40 (Tween-40) and polysorbate-80 (Tween-80). In another embodiment, the non-ionic surfactant is selected from the group consisting of polysorbate-20, polysorbate-40 and polysorbate-80. In one embodiment, the non-ionic surfactant comprises from about 2 to about 10% and any and all whole or partial increments there between, by weight of the solvent. In a further embodiment, the formulation comprises water and a non-ionic surfactant wherein the non-ionic surfactant comprises about 5% by weight of the solvent. In another embodiment, the formulation comprises water and a non-ionic surfactant wherein the non-ionic surfactant comprises about 5% by weight of the formulation. Polyethoxylated castor oils include, for example, Cremophors, such as polyethoxylated castor oil 35 (Cremophor RH40).

In one embodiment, the formulation comprises an anti-fungal drug in a therapeutically effective amount. A “therapeutically effective amount” is an amount of anti-fungal drug that is sufficient to prevent development of or alleviate to some extent one or more of a patient's symptoms of the disease being treated. In another embodiment, the formulation comprises an anti-fungal drug at a concentration sufficient to treat a fungal infection. In another embodiment, the anti-fungal drug is present at a concentration sufficient to treat a fungal infection of the nail. In yet another embodiment, the anti-fungal drug is present at a concentration sufficient to treat onychomycosis. In one embodiment, the antifungal drug is present at a concentration of at least about 5 mg/ml. In yet another embodiment, the antifungal drug is present at a concentration of at least about 10 mg/ml. In a further embodiment, the antifungal drug is present at a concentration of at least about 20 mg/ml.

In one embodiment, the formulation comprises terbinafine and the terbinafine is present in the formulation at a concentration sufficient to treat a fungal infection. In another embodiment, the terbinafine is present at a concentration sufficient to treat a fungal infection of the nail. In yet another embodiment, the terbinafine is present at a concentration sufficient to treat onychomycosis. In one embodiment, the terbinafine is present at a concentration of at least about 5 mg/ml. In another embodiment, the terbinafine is present at a concentration of at least about 10 mg/ml. In a further embodiment, the terbinafine is present at a concentration of at least about 25 mg/ml. In one embodiment, the terbinafine is present at a concentration from about 5 to about 100 mg/ml and any and all whole or partial increments there between. In yet another embodiment, the terbinafine is present at a concentration from about 5 to about 50 mg/ml and any and all whole or partial increments there between. In an additional embodiment, the terbinafine is present at a concentration from about 20 to about 50 mg/ml and any and all whole or partial increments there between. In a further embodiment, the terbinafine is present at a concentration of about 40 mg/ml.

In another embodiment, the terbinafine is present at a concentration from about 5 to about 25 mg/ml and any and all whole or partial increments there between. In another embodiment, the terbinafine is present at a concentration from about 5 to about 15 mg/ml and any and all whole or partial increments there between. In yet another embodiment, the terbinafine is present at a concentration from about 5 to about 12 mg/ml and any and all whole or partial increments there between. In a further embodiment, the terbinafine is present at a concentration from about 5 mg/ml to at least about 10 mg/ml and any and all whole or partial increments there between. In yet another embodiment, the formulation comprises a salt. In a further embodiment, the formulation comprises a phosphate buffered saline.

In an additional embodiment, the terbinafine is present in the composition in an amount between about 1 and about 10% (w/w) and any and all whole or partial increments there between. In another embodiment, the terbinafine is present in the composition in an amount between about 2 and about 6% (w/w) and any and all whole or partial increments there between. In yet another embodiment, the terbinafine is present in the composition in an amount between about 6 and about 10% (w/w) and any and all whole or partial increments there between. In a further embodiment, the terbinafine is present in an amount of about 4% (w/w).

In one embodiment, the formulation has a viscosity from about 100 to about 1000 cp at 25° C., and any and all whole or partial increments there between. In a further embodiment, the formulation of the invention comprises a viscosity modifying agent. A viscosity modifying agent can be added to the formulation to achieve the desired viscosity. A viscosity modifying agent includes any agent that is capable of modulating the viscosity of a gel. Viscosity modifying agents useful in the practice of the invention include but are not limited to, ionic and non-ionic, high viscosity, water soluble polymers; crosslinked acrylic acid polymers such as the “carbomer” family of polymers, e.g., carboxypolyalkylenes that may be obtained commercially; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose (HEC), hydroxypropyl methylcellulose, hydroxypropyl methylcellulose phthalate, methyl cellulose, carboxymethyl cellulose, and etherified cellulose; gums such as tragacanth and xanthan gum; sodium alginate, calcium alginate; gelatin, hyaluronic acid and salts thereof, chitosans, gellans or any combination thereof. It is preferred that non-basic viscosity enhancing agents, such as a neutral or acidic agent (such as, phosphoric acid or hydrochloric acid) be employed in order to facilitate achieving the desired pH of the formulation. If a uniform gel is desired, dispersing agents such as alcohol, sorbitol or glycerin can be added, or the gelling agent can be dispersed by trituration, mechanical mixing, or stirring, or combinations thereof. In one embodiment, the viscosity enhancing agent can also provide the acid, discussed above.

In one embodiment, the viscosity modifying agent is cellulose that has been modified such as by etherification or esterification. The pharmaceutically acceptable carrier or excipient may comprise about 0.1 to 10 weight percent of a viscosity modulating agent. The formulation of terbinafine hydrochloride can be a gel.

It is to be understood that the formulations of the present invention can comprise one or more of the components described above. In addition, it is to be understood that the formulations can comprise one or more of each type of component described above; for example, the formulation may comprise one or more solvent or co-solvents and/or one or more permeation enhancers.

In one embodiment, the one or more solvents or co-solvents are selected from the group consisting of water, an alcohol and glycerin. An exemplary alcohol is ethanol. In yet another embodiment, the formulation comprises one or more permeation enhancers selected from the group consisting of benzoic acid, polyethylene glycol and polyvinylpyrrolidone. In an additional embodiment, the formulation comprises benzoic acid. In a further embodiment, the formulation comprises benzoic acid and a polyethylene glycol. In an additional embodiment, the formulation comprises benzoic acid and polyethylene glycol 400 (PEG 400).

In an additional embodiment, the formulation comprises about 1 to about 10% (w/w) terbinafine hydrochloride, from about 10 to about 30% (w/w) ethanol (95% ethanol), and about 1 to about 40% polyethylene glycol 400, and any and all whole or partial increments there between.

In another embodiment, the formulation comprises about 1 to about 10% (w/w) terbinafine hydrochloride, from about 10 to about 30% (w/w) ethanol (95% ethanol), about 1 to about 50% glycerin and any and all whole or partial increments there between.

In yet another embodiment, the formulation comprises about 1 to about 10% (w/w) terbinafine hydrochloride, from about 10 to about 30% (w/w) ethanol (95% ethanol), about 1 to about 40% polyethylene glycol 400 and about 1 to about 50% glycerin and any and all whole or partial increments there between.

In an additional embodiment, the formulation comprises from about 1 to about 6% (w/w) terbinafine hydrochloride, from about 10 to about 30% (w/w) ethanol (95% ethanol) and from about 10 to about 50% (w/w) glycerin and any and all whole or partial increments there between.

In yet another embodiment, the formulation comprises from about 1 to about 6% (w/w) terbinafine hydrochloride, from about 10 to about 30% (w/w) ethanol (95% ethanol), from about 10 to about 50% (w/w) glycerin and from about 0.05 to about 0.5% (w/w) benzoic acid and any and all whole or partial increments there between.

In an additional embodiment, the formulation comprises about 4% (w/w) terbinafine hydrochloride, about 21% (w/w) ethanol (95% ethanol), about 40% (w/w) glycerin and about 0.2% (w/w) benzoic acid.

In yet another embodiment, the formulation comprises about 4% (w/w) terbinafine hydrochloride, about 21% (w/w) ethanol (95% ethanol), about 5% (w/w) polysorbate-80, and about 40% polyethylene glycol 400.

In a further embodiment, the formulation comprises about 4% (w/w) terbinafine hydrochloride, about 21% (w/w) ethanol (95% ethanol), about 5% (w/w) polysorbate-80, about 40% polyethylene glycol 400, 0.01% (w/w) BHT and about 0.01% EDTA.

In another embodiment, the formulation comprises about 4% (w/w) terbinafine hydrochloride, about 21% (w/w) ethanol (95% ethanol), about 5% (w/w) polysorbate-80, about 10% (w/w) glycerin, about 0.2% (w/w) benzoic acid and about 30% (w/w) polyethylene glycol 400.

In a further embodiment, the formulation comprises about 4% (w/w) terbinafine hydrochloride, about 21% (w/w) ethanol (of 95% ethanol), about 5% (w/w) polysorbate-80, about 10% (w/w) glycerin, about 0.2% (w/w) benzoic acid, about 40% (w/w) polyethylene glycol 400, 0.01% (w/w) BHT and about 0.01% EDTA.

In yet another embodiment, the formulation comprises about 4% (w/w) terbinafine hydrochloride, about 21% (w/w) ethanol (of 95% ethanol), about 5% (w/w) polysorbate-80, about 40% (w/w) glycerin, about 0.3% HEC, 0.2% (w/w) benzoic acid, about 0.01% (w/w) BHT and about 0.01% (w/w) disodium EDTA.

In an additional embodiment, the formulation comprises about 4% (w/w) terbinafine hydrochloride, about 21% (w/w) ethanol (of 95% ethanol), about 5% (w/w) polysorbate-80, about 2% (w/w) polyvinylpyrrolidone, 0.01% (w/w) BHT and about 0.01% EDTA.

In a further embodiment, the formulation comprises about 4% (w/w) terbinafine hydrochloride, about 21% (w/w) ethanol (95% ethanol), about 5% (w/w) polysorbate-80, about 30% (w/w) polyethylene glycol (PEG) 400, about 10% (w/w) glycerin and about 0.3% (w/w) HEC.

In yet another embodiment, the formulation comprises about 4% (w/w) terbinafine hydrochloride, about 21% (w/w) ethanol (95% ethanol), about 5% (w/w) polysorbate-80, about 30% (w/w) polyethylene glycol (PEG) 400, about 10% (w/w) glycerin, about 0.3% (w/w) HEC, 0.01% (w/w) BHT and 0.01% (w/w) disodium EDTA.

In an additional embodiment, the formulation comprises about 4% (w/w) terbinafine hydrochloride, about 21% (w/w) ethanol (95% ethanol), about 5% (w/w) polysorbate-80, about 30% (w/w) polyethylene glycol (PEG) 400, about 10% (w/w) glycerin, about 2% (w/w) polyvinylpyrrolidone and about 0.3% (w/w) HEC.

In an additional embodiment, the formulation comprises about 4% (w/w) terbinafine hydrochloride, about 21% (w/w) ethanol (95% ethanol), about 5% (w/w) polysorbate-80, about 30% (w/w) polyethylene glycol (PEG) 400, about 10% (w/w) glycerin, about 2% (w/w) polyvinylpyrrolidone, about 0.3% (w/w) HEC, about 0.01% (w/w) BHT and about (w/w) 0.01% disodium EDTA.

In yet another embodiment, the formulation comprises from about 5 to about 10% (w/w) terbinafine hydrochloride, 21% (w/w) ethanol (95% ethanol), 5% (w/w) polysorbate-80, about 30% (w/w) polyethylene glycol and 0.3% (w/w) HEC.

In a further embodiment, the formulation comprises from about 5 to about 10% (w/w) terbinafine hydrochloride, 21% (w/w) ethanol (95% ethanol), 5% (w/w) polysorbate-80, about 30% (w/w) polyethylene glycol, about 0.3% (w/w) HEC (w/w), about 0.01% (w/w) BHT and about 0.01% (w/w) EDTA.

### Methods of Treatment

As contemplated herein, the invention is directed to a method of administering an anti-fungal drug to a patient in need thereof comprising iontophoretically administering to a body surface of the patient a suitable formulation of the anti-fungal drug. In certain embodiments, the patient is a mammal, and preferably a human. In one embodiment, the invention is directed to a method of administering terbinafine to a patient in need thereof comprising iontophoretically administering to a body surface of the patient a suitable formulation of terbinafine. In one embodiment, the terbinafine is terbinafine hydrochloride. A formulation of the anti-fungal drug, such as terbinafine, may be administered to any affected body surface. Such body surfaces include, for example, the skin, the scalp, the groin, the feet, or the nails (fingernails and/or toenails). In one embodiment, the formulation is administered to the nail or to the nail and surrounding soft tissue and/or the nail and skin interface. In one embodiment, the formulation is administered to the nail or a portion of the nail with a masked drape applicator. In another embodiment, the formulation is administered to at least a portion of the nail and a portion of the surrounding tissue with a full drape applicator. In another embodiment, the method comprises pre-treating the nail such that delivery to the nail is increased. In another embodiment, the method comprises treating the nail with aqueous hydration, solvent or debridement before administering the anti-fungal drug to the nail. In one embodiment, the nail is pretreated with saline. In a further embodiment, the nail is pretreated with a formulation comprising one or more of the following excipients: a solvent including, but not limited to, dioxolane, dimethyl actamide, isopropyl alcohol (IPA) and ethyl acetate and/or a carrier agent including, but not limited to, an alcohol such as IPA. In one embodiment, the pretreatment formulation comprises a solvent in an amount up to about 25% (w/w). In another embodiment, the pretreatment formulation comprises a carrier agent in an amount up to about 20% (w/w).

In one embodiment, a current density sufficient for permeation of the formulation into a body surface is applied. In another embodiment, a current density sufficient for permeation into a nail plate is applied. In one embodiment, a current density from about 10 uA/cm² to about 1 mA/cm² is applied. In another embodiment, a current density from about 50 uA/cm² to about 1 mA/² is applied. In yet another embodiment, a current density from about 100 uA/cm² to about 500 uA/cm² is applied. In a further embodiment, a current density from about 200 uA/cm² to about 500 uA/cm² is applied. In yet another embodiment, a current density of at least about 200 uA/cm², about 400 uA/cm², about 600 uA/cm², about 800 uA/cm² or about 1 mA/cm² is applied. In a further embodiment, a current of no more than about 1 mA/cm² is applied. In another embodiment, a current of no more than about 750 uA/cm² is applied. In yet another embodiment, a current density of no more than about 500 uA/cm² is applied. Additional currents as described in the examples herein may also be applied.

In one embodiment, a current dose sufficient for permeation of the formulation into a body surface is administered. In an additional embodiment, a current dose of at least about 0.5 mA*min is applied. In yet another embodiment, a current dose of at least about 15 mA*min is applied. In a further embodiment, a current dose of at least about 25 mA*min is applied. In another embodiment, a current dose from about 0.5 mA*min to about 25 mA-min is applied. In a further embodiment, a current dose from about 1 mA-min to about 20 mA*min is applied. In yet another embodiment, a current dose from about 2 mA*min to about 15 mA-min is applied.

The iontophoresis can be applied for a sufficient time to achieve an effective amount of permeation. For example, a sufficient time for application is a time from about 1 minute to about 60 minutes. In one embodiment, iontophoresis is applied for a time from about 5 minutes to about 30 minutes. In yet another embodiment, iontophoresis is applied for a time from about 5 minutes to about 15 minutes. In a further embodiment, iontophoresis is applied for a time from about 10 minutes to about 30 minutes.

In one embodiment, the drug source is introduced into the nail using a mask, where terbinafine concentrations in peripheral areas of nail following iontophoretic treatment are less than drug concentrations directly under the mask. In a further embodiment, differences in delivered drug concentrations is effectuated by creating drug delivery gradients. In another embodiment, drug is delivered to dissimilar tissues based on different transfer efficiencies exhibited by the dissimilar tissues, resulting in higher percentage delivery in one tissue vs. another tissue. In a further embodiment, the concentrations of drug delivered in nail are greater than delivered to soft tissue. In yet a further embodiment, terbinafine is delivered into nail and nail bed vs. surrounding soft tissue, where transfer efficiencies are more than about 2 to 1 in favor of nail per mA-min (p=0.009).

In another embodiment, terbinafine is iontophoretically administered to the body surface at least twice. In a further embodiment, the terbinafine can be iontophoretically administered to the body surface at least three times. In a further embodiment, the terbinafine is iontophoretically administered to the body surface at least one time per week. In another embodiment, the terbinafine is iontophoretically administered at an interval from once a week to once every four weeks. In yet another embodiment, the terbinafine is iontophoretically administered at an interval from once a week to once a year. In a further embodiment, the terbinafine is iontophoretically administered at an interval from once a week to once every six months. In an additional embodiment, the terbinafine is administered at an interval from once a week to once every sixteen weeks. In a further embodiment, the terbinafine is administered an interval from once a week to once every twelve weeks. In another embodiment, the terbinafine is administered to the body surface at an interval from once every two weeks to once every eight weeks. In yet another embodiment, the terbinafine is administered to a body surface at an interval from once every week to once every four weeks. In an additional embodiment, the terbinafine is administered to a body surface at an interval from once every four weeks to once every eight weeks. In a further embodiment, the terbinafine is administered to the body surface at an interval from once every three weeks to once every four weeks.

In one embodiment, a formulation of anti-fungal drug is administered using an iontophoretic delivery device, including an applicator, as contemplated herein. In another embodiment, the formulation is adsorbed into a foam material and applied to the body surface. In yet another embodiment, the formulation is preloaded into the applicator and distributed as a single use, single dose applicator for administration using an iontophoretic delivery device.

In yet another embodiment, the invention is a method of treating a fungal infection in a patient suffering therefrom comprising iontophoretically administering a formulation comprising an antifungal drug. In one embodiment, the antifungal drug is terbinafine. In another embodiment, the terbinafine is terbinafine hydrochloride. In another embodiment, the fungal infection is a dermatophyte infection. In yet another embodiment, the fungal infection is selected from the group consisting of interdigital-type pedis, tinea curis, tinea capitis, tinea corporis, tinea versicolor and onychomycosis. Interdigital-type pedis is commonly known as athlete's foot and is a fungal infection of the foot.

In yet another embodiment, the fungal infection is a fungal infection of the nail. In a further embodiment, the fungal infection is onychomycosis. Onychomycosis is an invasion of the nail plate and nail bed by a fungus. In one embodiment, the invention is a method of treating onychomycosis in a patient suffering therefrom comprising iontophoretically administering a formulation comprising terbinafine hydrochloride to a nail plate of the patient.

In a further embodiment, the fungal infection is tinea capitis. In one embodiment, the invention is a method for the treatment of tinea capitis in a patient suffering therefrom comprising iontophoretically administering a formulation comprising terbinafine to the patient.

The methods described herein are by no means all-inclusive, and further methods to suit the specific application will be apparent to the ordinary skilled artisan. Moreover, the effective amount of the compositions can be further approximated through analogy to compounds known to exert the desired effect.

### EXAMPLES

The invention is now described with reference to the following Examples. These Examples are provided for the purpose of illustration only, and the invention is not limited to these Examples, but rather encompasses all variations which are evident as a result of the teachings provided herein.

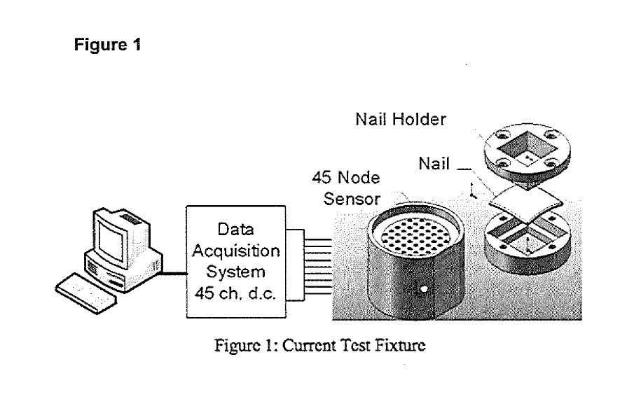

The results of drug loading experiments in cadaver toes reported by Nair [Nair et al., 2009, Pharm Res. 26(9):2194-201] were compared with current measurements made using the current test fixture of FIG. 1 and models for the two tissue groups. The experiments disclosed herein were conducted to determine 1) if a drug delivery gradient existed in masked drape applications, and how that correlated with drug assays in cadaver toes; and 2) to determine the proportion of current flowing in soft tissue vs. into nail in cadaver toe delivery, and to compare that with drug loading found by Nair in the two tissue types. It is believed that the transfer efficiency is higher in nail than into soft tissue.

The materials and methods employed in the experiments and examples disclosed herein are now described.

Current Distribution Testing: Cadaver nails were obtained for current distribution testing using the test fixture depicted in FIG. 1. This device is designed to support cadaver nail over agarose (skin analogue) and measure current distribution in two dimensions during iontophoresis using the masked drape and full drape applicator designs. It measures and records current flow in real time using a 45 node sensor with 3 mm spacing and associated data acquisition system. The overall fixture covers about a 12-20 mm diameter circle. Each nail was soaked in physiologic saline for approximately one hour prior to each treatment. The nails were placed on top of approximately 5 mm thick agarose gel of about 1% by weight Type II agarose and 20 mM saline, and exposed to iontophoretic treatments of terbinafine at about 0.5 mA for 5 min for the masked drape tests. For full drape testing, the nail was placed on top of agarose with agarose added on one end to simulate the cuticle area.

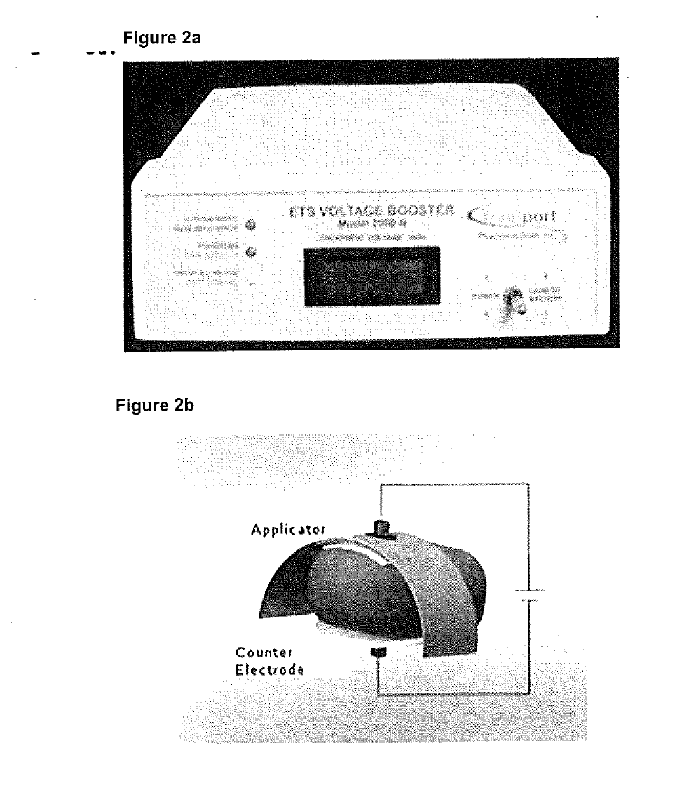

Cadaver Toe Delivery Testing: Six cadaver toes were soaked in physiologic saline prior to treatment. Each terbinafine loaded applicator type (masked drape and full drape) was placed on the toe and iontophoretic treatment of 0.5 mA for 20 min was delivered. Post treatment, the nails, and sections of nail bed and surrounding tissue, were excised and analyzed. The amount of loaded terbinafine was compiled for each section. The iontophoretic power source and general configuration for the applicator on the cadaver toe are depicted in FIGS. 2A and 2B. Additional details regarding methods used and experimental details for cadaver toe delivery tests can be found in [Nair et al., 2009, Pharm Res. 26(9):2194-201].

Both applicator designs were used in clinical studies, including clinical nail samples at various iontophoretic dose levels taken 24 hours post treatment. Table I summarizes the data compiled from the study. For an average dose of about 8 mA-min, drug amounts for masked drape clipping assays averaged about 423 ug/g and full drape clippings averaged about 842 ug/g. The clipping sizes and volumes varied (n=80, mean=3.1 mL, sd=1.8). The amount of drug found in nail clippings for the masked drape treatments appeared to be lower than expected given the higher current concentration in nail vs. levels found for the full drape treatments at a lower level of nail current.

TABLE 1
Phase 1 Clinical Data

| | | | | Mean Nail Levels (μg/g) | |
|---|---|---|---|---|---|
| | | | | Applicator Design | |
| | | Treatment | Treatment | Nail Only | Nail/Skin |
| Group # | Dose | Current | Duration | (N) | (N) |
| 1 | 3 mA-min | 0.3 mA | 10 min | 201 (8) | 631 (8) |
| 2 | 6 mA-min | 0.5 mA | 12 min | 600 (7) | 736 (8) |
| 3 | 6 mA-min | 0.3. mA | 20 min | 638 (5) | 988 (8) |
| 4 | 10 mA-min | 0.5 mA | 20 min | 492 (7) | 970 (7) |
| 5 | 15 mA-min | 0.5 mA | 30 min | 185 (4) | 886 (4) |

The results of the experiments presented herein are now described.

### Example 1

### Masked Drape

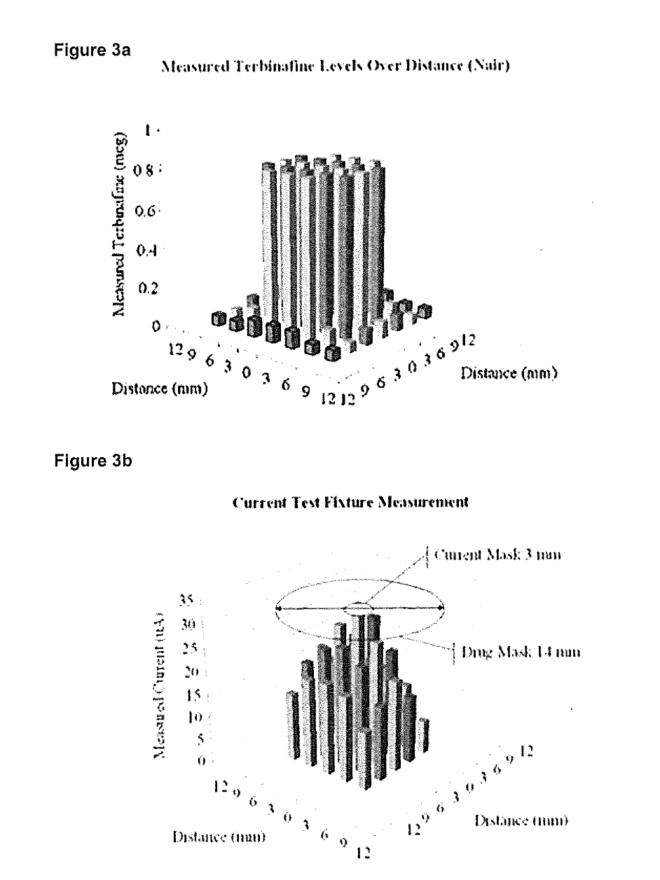

FIG. 3A depicts drug assay results from cadaver nails following iontophoresis using a masked drape applicator, while FIG. 3B depicts current measurements taken at the same current levels through cadaver nail into agarose. Terbinafine sample data was compiled allocating samples to the same relative spacing as current sensing nodes in the current test fixture of FIG. 1. Notably, a gradient of both current and drug is evident, resulting from iontophoretic delivery through the mask opening into the larger conductive medium of nail and underlying tissue. Results of finite element analysis for essentially masked electrodes over tissue that illustrate gradients with higher resolution can also be seen as provided by Kresteva and Papazov [Krasteva et al., Biomed Eng Online. 2002 Dec. 16; 1:7].

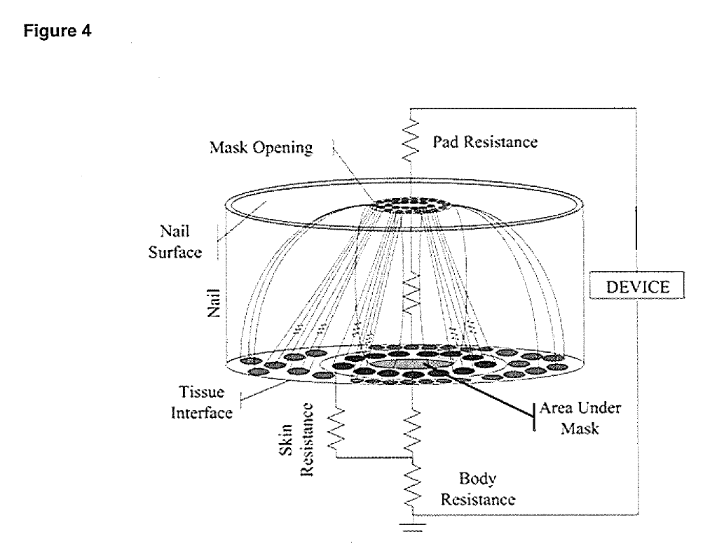

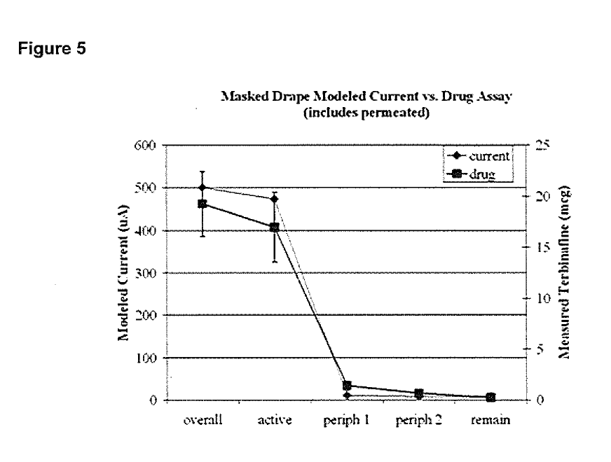

A piecewise linear analysis was performed in lieu of a finite element analysis (FEA) by approximating volume current flow using parallel resistances within each drug sample volume. The areas of each drug sample were estimated based upon the relative weight of the samples to the overall weight and dimensions of the nail. FIG. 4 depicts the relative sample areas and a simple illustration of the model used. A series of resistances (>150 per nail) were modeled based upon the thickness and surface areas of samples taken from cadaver toes. The current flowing through each resistance was summed per area to create an estimate of current distribution. The results of current calculated using the model, along with drug assay data, are depicted in FIG. 5.

### Example 2

### Full Drape

For the full drape applicator, the conductivity of soft tissue is in parallel with the conductivity of nail. Parallel conducting tissues have been modeled as parallel resistances. The amount of applied current that flows in each may be estimated as the relative percentage of one of those resistances to the sum of both as:

iₛₖᵢₙ=i_{applied}×rₙₐᵢₗ/(rₙₐᵢₗ+rₛₖᵢₙ), (1)

where i is current and r is resistance.

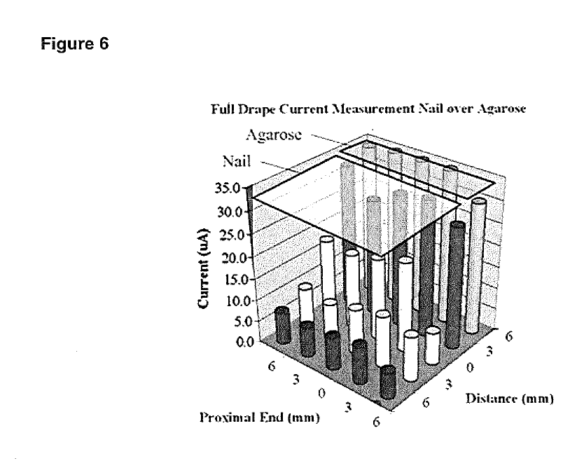

The current sensing test fixture of FIG. 1 was used to measure current flow as terbinafine was delivered from a full drape applicator to a cadaver nail. The nail was placed on top of agarose which extended to the side of one end of the nail (simulating the cuticle area and full drape coverage of nail and skin). The resulting current flow measurement is depicted in FIG. 6. Approximately 0.17 mA was measured in the nail vs. 0.33 mA measured in the skin (or approximately 34% nail, 66% skin).

Voltages measured during masked drape cadaver experiments were used to establish intrinsic nail impedance using the formula

ρ=r×A/h, (2)

where r=resistance in k-ohms, A=nail area in cm² and h=nail thickness in cm. An intrinsic impedance for nail (n=3) was found to be approximately 2753 k-ohm-cm, sd=217 from the cadaver toe data. The intrinsic impedance was paired with nail size and thickness measurements for the full drape applicators, solving for r, yielding an approximate average of Rₛₖᵢₙ=64 k-ohms and an approximate average of Rₙₐᵢₗ=152 k-ohms. The resistance ratio results in a ratio of current in nail and skin of about 30% nail, 70% skin.

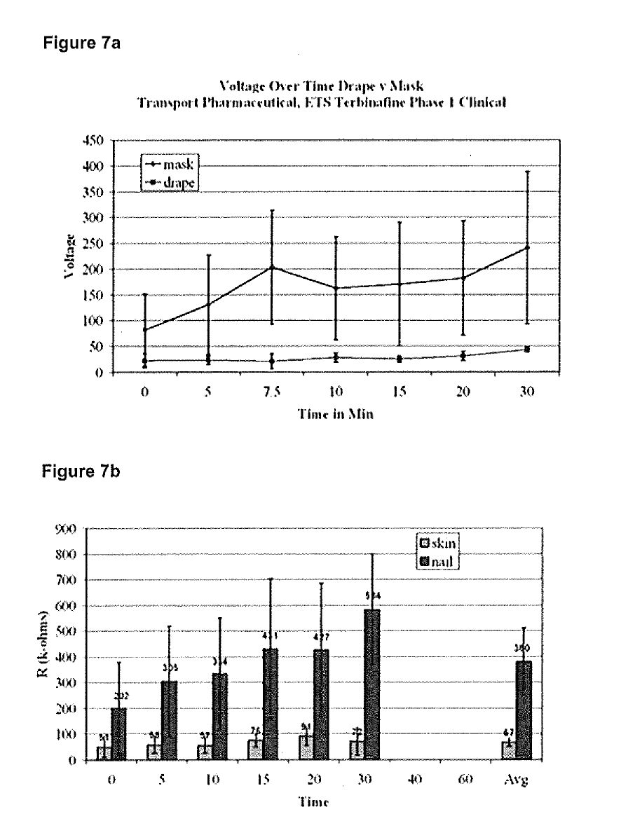

FIG. 7A depicts voltage measurements taken and the resulting calculated resistances (FIG. 7B) for skin and nail for full drape and masked drape treatments during clinical studies. The average skin resistance was about 67 k-ohms and nail resistance varied during treatment between about 202 k-ohms and 584 k-ohms. At time 0 (resistance fluctuates over time), the ratio in-vivo is about 25% nail, 75% skin (n=26).

Table II summarizes the relative resistances and current delivery ratios for nail/agarose, cadaver nail/skin and in-vivo nail/skin. As demonstrated in Table II, treatment area and thickness for skin, nail or agarose, as well as hydration levels, vary the impedances.

TABLE II
Current Delivery Ratios

| | Agarose/Nail | Cadaver | |
|---|---|---|---|
| | (k-ohms) | (k-ohms) | In-vivo (k-ohms) |
| Skin or Agarose | 10.7 | 64 | 67 |
| Nail | 20.7 | 152 | 202-584 |
| Current Ratio | 1.9:1 | 2.33:1 | 3-9 |
| Skin/Nail | (66%, 34%) | (70%, 30%) | (75%, 25%)-(90%, 10%) |

It is believed that the lower drug levels found during the clinical study from nail clippings in the masked drape applications was the result of samples at the extremities of the nail. As contemplated herein, the presence of a drug delivery gradient is supported by the location of clinical nail samples, the results of current distribution modeling and the cadaver toe experiments.

For example, the current measurement and drug loading maps for cadaver nail and toe as depicted in FIG. 3 indicate a significant gradient. Drug loading at the periphery of the nail was about 26 to 77 times less than drug loading in the center of the nail. Nail clippings for the masked drape group in the clinical studies provided herein were taken about 24 hours post treatment from the distal nail. Samples varied in size and volume, but averaged about 1.8 mm long. This size and location corresponds with the nail sampling performed in the cadaver toe experiments in the category of “peripheral 2” or “remaining” and depicted in FIG. 3A in about the 9 to 12 mm range. The current levels predicted by the model in the same areas correlate well with drug distribution samples, r=0.998, p=0.000.

From the full drape cadaver toe experiments, the amount of current expected to flow in the cadaver skin based upon calculated values of Rₛₖᵢₙ and Rₙₐᵢₗ are about 70% skin, 30% nail (See Table II). Current ratios for the clinical measurements are similar at treatment time. If terbinafine delivery into dissimilar tissues were strictly related to current ratio (the same efficiency of delivery into both tissue types), then the relative ratio of delivered terbinafine in skin and nail would be about 2.33 to 1 (or about 70% skin/30% nail).

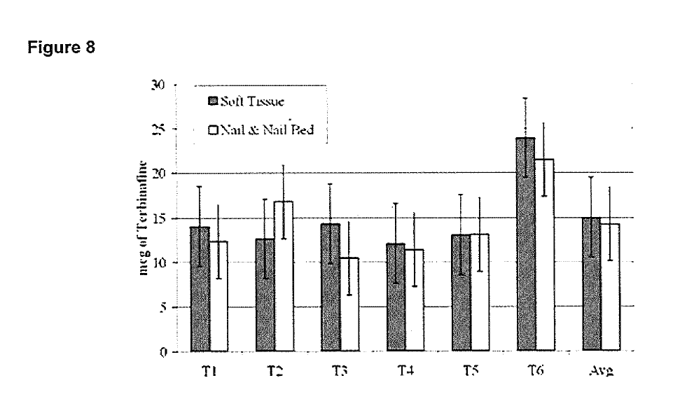

Extracted results illustrate statistical equivalence (p=0.78, n=6) of delivery into skin and nail & nail bed, as depicted in FIG. 8, suggesting that there was a difference in drug loading efficiency between the two tissue types.

Other studies have reported similar differences in the effect of tissues and electro-chemical influences on drug uptake during iontophoresis. For example, L. Wearley [Nair et al., 2009, Pharm Res. 26(9):2194-201] studied the effect of binding on the iontophoretic transport of amino acids in skin. In this study, it was demonstrated that the stratum corneum exhibits binding behavior which retards iontophoretic delivery of amino acids. It is believed that differences in keratin content, lipid content combined with pre-soaked nail acting as a porous hydrogel, may enhance hydrophilic drug formulation transfer characteristics; however this has not been studied specifically.

D. de Berker [2007, Int J Cosmet Sci. 29(4):241-75] in an article on nail biology, indicates that healthy nail is 1000 times more permeable to water than skin. This property of nail may influence the electroosmotic element of iontophoretic drug delivery in nail and further differentiate the two tissue types from a drug delivery perspective. In an analysis of the process of electromigration and electroosmosis of molecules into tissue, shows that molecular transfer does not follow a simple 1:1 ratio with current. Rather, the efficiency of transfer is dependent upon many factors including charge mobility and drug concentration (not to mention time varying effects of pH or local charge concentrations). In addition, Kalia notes apparent saturation effects at higher current levels related to physiochemical properties.

As contemplated herein, overall, different drug transfer efficiencies should be considered when delivering into dissimilar tissues simultaneously with the full drape applicator. Using Kalia's analysis as an example. Drug flux is:

Jₓ^{T}=Jₓ^{EM}+Jₓ^{EO}=(1/zₓAF)×iₓ×i+vcₓ (3)

where tₓ is the transport number, F is Faraday's constant, A is area of application and zₓ is charge. For a particular “membrane” or “tissue type”, tₓ is the ratio of (charge, mobility and drug concentration of the charge carrier) to the product of these for all charge carriers, i is current and vcₓ is an electroosmotic term. Rather than determine individual elements of Kalia's formula, an aggregate transport number of skin (tₛₖᵢₙ) and nail & nail bed (tₙₐᵢₗ) can be calculated, essentially aggregating the factors and segmenting them into soft tissue plus nail/nail bed.

Jₓ^{T}=Jₓ^{Tskin}+Jₓ^{Tnail}=(1/zₛₖᵢₙAF)tₛₖᵢₙiₛₖᵢₙ+vcₛₖᵢₙ+(1/zₙₐᵢₗAF)tₙₐᵢₗiₙₐᵢₗ+vcₙₐᵢₗ=lₛₖᵢₙ+lₙₐᵢₗ (4)

Where l is the amount of drug loaded for skin or nail. For a given dose in uA-min, the amount of terbinafine loaded into cadaver nail & nail bed vs. cadaver skin vs. delivered dose and calculated transfer number is

lₜₒₜₐₗ=lₛₖᵢₙ+lₙₐᵢₗ,lₛₖᵢₙ=dₛₖᵢₙ×tₛₖᵢₙ and lₙₐᵢₗ=dₙₐᵢₗ×tₙₐᵢₗ in ug/mA-min, (5)

where dₛₖᵢₙ, dₙₐᵢₗ is the product of current through skin (or nail) and time (dose), tₛₖᵢₙ, tₙₐᵢₗ is an aggregate of all factors related to delivery into skin (or nail) and lₛₖᵢₙ, lₙₐᵢₗ is the amount of terbinafine loaded into skin (or nail).

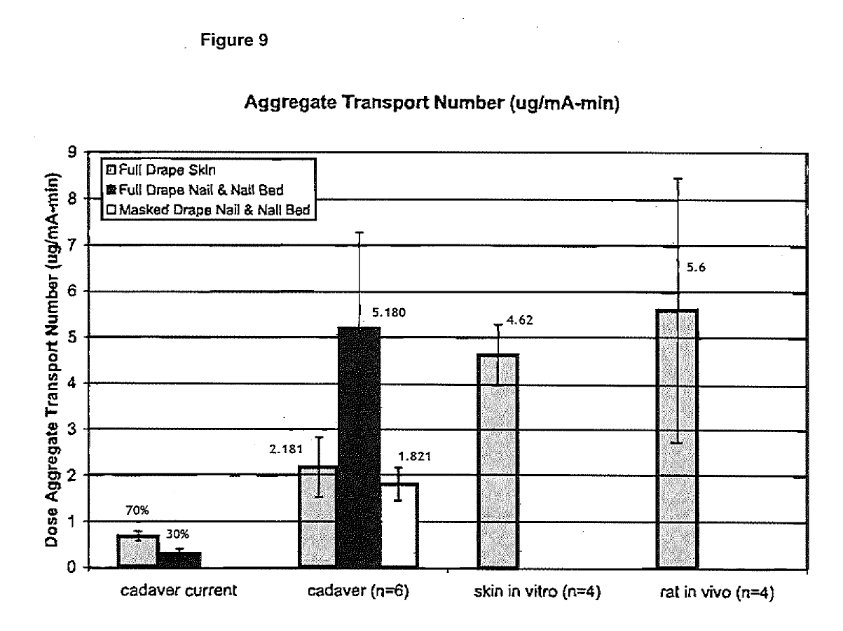

Table III lists the areas of delivery, current and transfer number calculations for the 6 cadaver toe trials. FIG. 9 depicts the average transport numbers including values calculated for current (for reference), skin alone in Franz cell and Rat.

TABLE III
Drug per unit area vs. current per unit area (from Cadaver Toe)

| current density | Trial 1 | Trial 2 | Trial 3 | Trial 4 | Trial 5 | Trial 6 | Avg | sd |
|---|---|---|---|---|---|---|---|---|
| skin area (cm2) | 3.2 | 3.53 | 3.37 | 2.85 | 3.05 | 3.73 | 3.288 | 0.322 |
| nail area (cm2) | 1.8 | 2.72 | 2.88 | 3.4 | 3.2 | 2.52 | 2.753 | 0.565 |
| skin idensity (mA/cm2) | 0.126 | 0.110 | 0.108 | 0.089 | 0.109 | 0.094 | 0.106 | 0.013 |
| nail idensity (mA/cm2) | 0.054 | 0.042 | 0.048 | 0.072 | 0.052 | 0.059 | 0.054 | 0.010 |
| skin density dose (mA-min/cm²) | 2.518 | 2.192 | 2.151 | 1.788 | 2.181 | 1.885 | 2.119 | 0.258 |
| nail density dose (mA-min/cm²) | 1.080 | 0.831 | 0.956 | 1.442 | 1.047 | 1.178 | 1.089 | 0.209 |
| tf skin = (ug/cm²)/(dose/cm²) | 1.745 | 1.632 | 1.975 | 2.368 | 1.961 | 3.403 | 2.181 | 0.650 |
| tf nail = (ug/cm²)/(dose/cm²) | 6.358 | 7.438 | 3.805 | 2.328 | 3.917 | 7.237 | 5.180 | 2.114 |

For cadaver toe with the full drape application, the ratio of skin to nail aggregate transport number is about 1 to 2.38. Thus, transfer to nail is about 2.38 times more per mA-min than into skin (n=6, p=0.009). This contrasts with a current “delivery” ratio of 2.3 to 1 (2.3 times more current in skin than in nail). Interestingly, the aggregate transport number for the nail-only application is less than the factor for nail in the full drape application (n=4, p=0.032). Therefore, it is possible that the decreased nail treatment area of the masked drape application reduces terbinafine loading vs. the applicator with an increased coverage area.

Applying the cadaver transfer number for nail vs. skin to the in-vivo results yields an estimate for nail and drug delivered for both tissues (based upon resistance ratios calculated from measured voltages during the clinical study.)

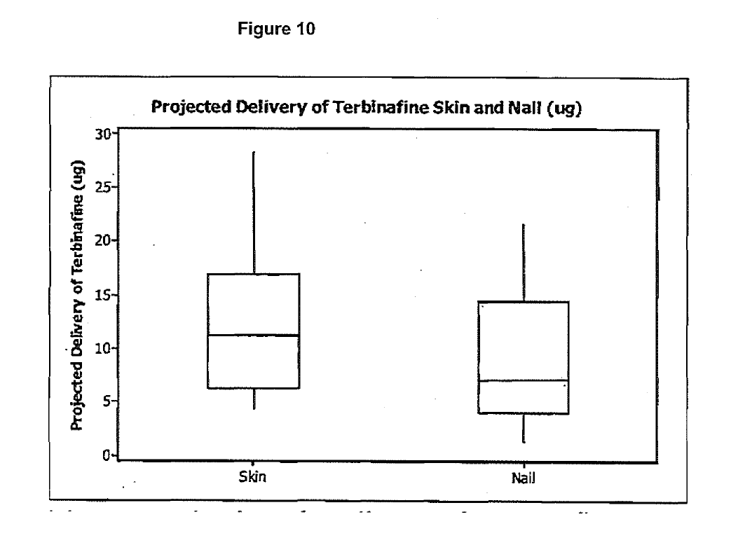

FIG. 10 depicts the projected delivery of terbinafine into skin and nail for full drape using cadaver transfer number calculation. The mean level of terbinafine estimated to have been delivered with full drape treatments to patients in the clinical studies using this method is about 12.4 ug skin, 9.4 ug nail, as compared with a mean of about 15 ug skin and 14 ug nail for cadaver toes.

Thus, terbinafine concentrations in peripheral areas of nail following iontophoretic treatment, where the drug source is introduced into the nail using a mask, were shown to be less than drug concentrations directly under the mask due to current and drug delivery gradients. Terbinafine concentrations resulting from simultaneous iontophoretic delivery to dissimilar tissues were also found to exhibit different transfer efficiencies, resulting in higher percentage delivery in nail than soft tissue. Notably, for terbinafine into nail and nail bed vs. surrounding soft tissue, transfer efficiencies were found to be more than about 2 to 1 in cadaver toes in favor of nail per mA-min (p=0.009).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method for treating a fungal infection of the nail of a patient suffering therefrom, comprising iontophoretically administering terbinafine to at least a portion of the infected nail, wherein the amount of terbinafine delivered is based upon a drug delivery gradient across the infected nail.

2. The method of claim 1, wherein the iontophoretic administration comprises a mask for limiting the nail surface to which terbinafine is iontophoretically administered.

3. The method of claim 1, wherein the drug delivery gradient is calculated prior to administration of terbinafine.

4. The method of claim 3, wherein the amount of current used to iontophoretically administer terbinafine is based on the calculated drug delivery gradient.

5. The method of claim 4, wherein the length of time and frequency of iontophoretic administration is based on the amount of current used.

6. The method of claim 1, wherein the terbinafine is a component of a formulation comprising terbinafine hydrochloride.

7. The method of claim 6, wherein the formulation further comprises one or more solvents or co-solvents and a permeation enhancer.

8. The method of claim 7, wherein the terbinafine hydrochloride is present in an amount from about 1 to about 10% (w/w).

9. The method of claim 7, wherein the one or more solvent or co-solvents are each present in an amount from about 10 to about 50% (w/w).

10. The method of claim 9, wherein the formulation further comprises water, an alcohol and glycerin.

11. A method for treating a fungal infection of the nail of a patient suffering therefrom, comprising iontophoretically administering terbinafine to at least a portion of the nail and at least a portion of tissue adjacent the nail, wherein the amount of terbinafine delivered to the nail and adjacent nail tissue is based upon different transfer efficiencies of the nail and adjacent tissue, such that the nail receives a higher percentage delivery of terbinafine than the adjacent tissue.

12. The method of claim 11, wherein the different transfer efficiencies of the nail and adjacent tissue is calculated prior to administration of terbinafine.

13. The method of claim 12, wherein the amount of current used to iontophoretically administer terbinafine is based on the calculated transfer efficiencies of the nail and adjacent tissue.

14. The method of claim 13, wherein the length of time and frequency of iontophoretic administration is based on the amount of current used.

15. The method of claim 11, wherein the terbinafine is a component of a formulation comprising terbinafine hydrochloride.

16. The method of claim 15, wherein the formulation further comprises one or more solvents or co-solvents and a permeation enhancer.

17. The method of claim 15, wherein the terbinafine hydrochloride is present in an amount from about 1 to about 10% (w/w).

18. The method of claim 16, wherein the one or more solvent or co-solvents are each present in an amount from about 10 to about 50% (w/w).

19. The method of claim 18, wherein the formulation further comprises water, an alcohol and glycerin.

20. The method of claim 11, wherein the ratio of the different transfer efficiencies in the nail and adjacent nail tissue are equal to or greater than about 2 to 1.

21. A method for treating a fungal infection of the nail of a patient suffering therefrom comprising:
calculating the drug delivery gradient across at least a portion of the nail of the patient; and
iontophoretically administering terbinafine to at least a portion of the infected nail, wherein the terbinafine is delivered at a concentration based upon the calculated drug delivery gradient.

22. A method for treating a fungal infection of the nail of a patient suffering therefrom comprising:
calculating the drug transfer efficiency of at least a portion of the nail and the adjacent nail tissue; and
iontophoretically administering terbinafine to at least a portion of the nail and at least a portion of the tissue adjacent the nail, wherein the terbinafine is delivered to the nail and adjacent nail tissue based upon the calculated transfer efficiencies of the nail and adjacent tissue, such that the nail receives a higher percentage delivery of terbinafine than the adjacent tissue.
